(19) United States
(12) Patent Application Publication
(10) Pub. No.: US 2024/0407652 A1
(43) Pub. Date: Dec. 12, 2024

(54) TEMPERATURE MEASURING DEVICE

(71) Applicant: Nippon Telegraph and Telephone Corporation, Tokyo (JP)

(72) Inventors: Yujiro Tanaka, Tokyo (JP); Daichi Matsunaga, Tokyo (JP)

(21) Appl. No.: 18/689,649

(22) PCT Filed: Oct. 21, 2021
(86) PCT No.: PCT/JP2021/038900

§ 371 Date: Mar. 6, 2024

(51) Int. Cl. A61B 5/01

(52) U.S. Cl. CPC A61B 5/01

(57) ABSTRACT

A temperature measurement device includes: a thermal conductor having a hollow structure in which a peripheral edge portion thereof is disposed in contact with a living body and which has thermal conduction anisotropy in which a thermal conductivity in an in-plane direction is higher than a thermal conductivity in a thickness direction and flexibility; a heat insulating material disposed to fill a space between the living body and the thermal conductor and having flexibility; sensors that measures a magnitude of a heat flow transmitted from the living body; a heat insulating material disposed to cover the thermal conductor and having flexibility; and an electronic circuit that calculates an internal temperature of the living body on the basis of the measured magnitude of the heat flow.

### CROSS-REFERENCE TO RELATED APPLICATIONS

This application is a national phase entry of PCT Application No. PCT/JP2021/038900, filed on Oct. 21, 2021, which application is hereby incorporated herein by reference.

### TECHNICAL FIELD

The present invention relates to a temperature measurement device that noninvasively and accurately measures an internal temperature of a living body.

### BACKGROUND

Recent research on time biology has revealed that circadian rhythms, so-called body clocks, possessed by humans are closely related to various things related to the human body, such as not only the quality of sleep, exercise, and work, but also the effect of medication and the onset of disease. A circadian rhythm is almost a constant rhythm, but it is known that the circadian rhythm greatly changes depending on light exposure in daily life, exercise, dietary habits, and age and sex.

A core body temperature is known as an index for measuring a circadian rhythm. However, in general, a method of measuring a core body temperature is a method of inserting a thermometer into the rectum or measuring the temperature of the eardrum in a state in which the ears are sealed, and is a very stressful method as a method of measuring a core body temperature during daily activities or during sleep.

Meanwhile, as a technique for noninvasively measuring the core body temperature of a living body, a technique for estimating the core body temperature of the living body by replacing the flow of heat in a pseudo manner with a one-dimensional equivalent circuit model has been proposed (see NPL 1).

The method disclosed in NPL 1 estimates a core body temperature T_{cbt} of a living body 100 using a thermal equivalent circuit model of the living body 100 and a sensor 101 as illustrated in FIG. 9. When the sensor 101 having a thermal resistance Rₛₑₙₛₒᵣ is placed on the surface of the living body 100, the core body temperature T_{cbt} of the living body 100 can be estimated from the temperature Tₛₖᵢₙ of the skin surface of the living body 100 and the temperature Tₜₒₚ of the upper surface of the sensor 101 on the side opposite to the surface in contact with the living body 100 using Expression (1).

$${T}_{c b t} = {T}_{s k i n} + α \times ( {T}_{skin} - {T}_{t o p} ) \qquad ( 1 )$$

Alternatively, the core body temperature T_{cbt} can be estimated from the heat flux Hₛₖᵢₙ of the skin surface of the living body 100 as represented by Expression (2).

$${T}_{c b t} = {T}_{s k i n} + α \times {H}_{s k i n} \qquad ( 2 )$$

In Expressions (1) and (2), a is a proportionality coefficient related to the thermal resistance R_{body} of the living body 100. The proportionality coefficient α can be calibrated in advance by another measurement means for measuring the eardrum temperature, the rectum temperature, and the like.

However, in the method of estimating the core body temperature T_{cbt} using Expressions (1) and (2), in a case where the outside air temperature changes or wind hits the living body 100, the heat flow is not one-dimensional, the heat that should flow into the sensor 101 flows out to the surroundings, the magnitude of the heat flow which should be measured decreases and thus there is a problem that an error occurs in estimation of the core body temperature T_{cbt}. For this reason, there is a possibility that application to a core body temperature monitor in daily life would be difficult due to limitation of use in a limited environment in a hospital.

Therefore, in order to reduce an estimation error of the core body temperature T_{cbt}, the inventors have proposed a sensor structure in which a one-dimensional heat flow is obtained even if there is a surrounding environmental change in NPL 1. In this structure, a temperature sensor is covered with a truncated cone-shaped or dome-shaped metal member including aluminum or the like having a high thermal conductivity, and thus the temperature of the surroundings is increased with respect to the central portion where the temperature sensor is located, thereby reducing the flow (loss) of heat to the surroundings. As a result, an estimation error of the core body temperature T_{cbt} can be reduced.

However, in the method disclosed in NPL 1, a temperature measurement device becomes hard without allowing shape change because the metal member is used, and thus there is a possibility that the temperature measurement device would not be able to be attached to a human body having a complicated curved surface. In addition, a feeling of wearing is poor, and there is a possibility that a person wearing the device would be injured by the hard device. Furthermore, in the case of a metal such as aluminum, heat transfer is isotropic, and thus there is a problem that heat transport from a central portion where a temperature sensor is provided to the surroundings cannot be curbed.

### CITATION LIST

### Non Patent Literature

Non Patent Literature 1: Y. Tanaka, D. Matsunaga, T. Tajima, and M. Seyama, “Robust Skin Attachable Sensor for Core Body Temperature Monitoring”, IEEE SENSORS JOURNAL, VOL. 21, NO. 14, pp. 16118-16123, Jul. 15, 2021

### SUMMARY

### Technical Problem

Embodiments of the present invention have been made to solve the above problems, and an object of embodiments of the present invention is to provide a temperature measurement device that can be worn on various parts of a living body, can improve a feeling of wearing on the living body, and can accurately measure an internal temperature of the living body.

### Solution to Problem

A temperature measurement device of embodiments of the present invention includes: a thermal conductor having a hollow structure, a peripheral edge portion of the thermal conductor being disposed in contact with a living body, the thermal conductor having thermal conduction anisotropy in which a thermal conductivity in an in-plane direction is higher than a thermal conductivity in a thickness direction and flexibility; a first heat insulating material having flexibility, the first heat insulating material being disposed to fill a space between the living body and the thermal conductor; a sensor provided on the first heat insulating material to measure a magnitude of a heat flow transmitted from the living body; a second heat insulating material having flexibility, the second heat insulating material being disposed to cover the thermal conductor; and an electronic circuit configured to calculate an internal temperature of the living body on the basis of the magnitude of the heat flow measured by the sensor.

Further, a configuration example of the temperature measurement device of embodiments of the present invention is characterized in that the electronic circuit is provided inside the second heat insulating material.

Further, a configuration example of the temperature measurement device of embodiments of the present invention is characterized in that the electronic circuit is provided at a plurality of locations in a distributed manner inside the second heat insulating material.

Further, a configuration example of the temperature measurement device of embodiments of the present invention is characterized in that a space is formed inside the second heat insulating material.

Further, a configuration example of the temperature measurement device of embodiments of the present invention is characterized in that a plurality of the thermal conductors and a plurality of the second heat insulating materials are alternately laminated.

Further, a configuration example of the temperature measurement device of embodiments of the present invention is characterized by further including a film for preventing radiation of heat provided to cover the second heat insulating material on the outer side.

Further, a configuration example of the temperature measurement device of embodiments of the present invention is characterized in that the sensor includes a first temperature sensor provided on a surface of the first heat insulating material facing the living body and configured to measure a temperature of a surface of the living body, and a second temperature sensor configured to measure a temperature inside the first heat insulating material immediately above the first temperature sensor, and the electronic circuit calculates the internal temperature of the living body on the basis of measurement results of the first and second temperature sensors.

### Advantageous Effects of Embodiments of Invention

According to embodiments of the present invention, by providing the thermal conductor, the internal temperature of a living body can be accurately measured even when the convection state of the outside air changes. Further, in embodiments of the present invention, by providing flexibility to the thermal conductor and the first and second heat insulating materials, it is easy to attach the temperature measurement device to various parts of a living body. In addition, the feeling of wearing on a living body can be improved, and the possibility of the living body being injured by the hard device can be reduced.

### BRIEF DESCRIPTION OF THE DRAWINGS

FIG. 1 is a diagram illustrating a configuration of a temperature measurement device according to a first embodiment of the present invention.

FIG. 2 is a flowchart for describing the operation of the temperature measurement device according to the first embodiment of the present invention.

FIG. 3 is a diagram illustrating a core body temperature estimated by the temperature measurement device according to the first embodiment of the present invention and an eardrum temperature measured by an eardrum thermometer.

FIG. 4 is a diagram illustrating temporal changes in a core body temperature estimated by the temperature measurement device according to the first embodiment of the present invention and an eardrum temperature measured by the eardrum thermometer.

FIG. 5 is a diagram illustrating a configuration of a temperature measurement device according to a second embodiment of the present invention.

FIG. 6 is a diagram illustrating a configuration of a temperature measurement device according to a third embodiment of the present invention.

FIG. 7 is a diagram illustrating a configuration of a temperature measurement device according to a fourth embodiment of the present invention.

FIG. 8 is a block diagram illustrating a configuration example of a computer that realizes the temperature measurement devices according to the first to fourth embodiments of the present invention.

FIG. 9 is a diagram illustrating a thermal equivalent circuit model of a living body and a sensor.

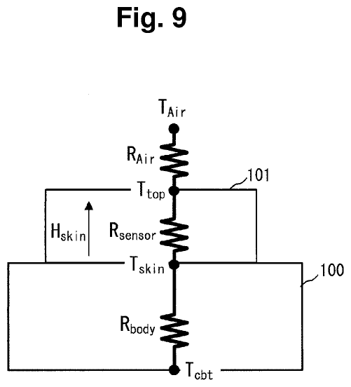

### DETAILED DESCRIPTION OF ILLUSTRATIVE EMBODIMENTS

### First Embodiment

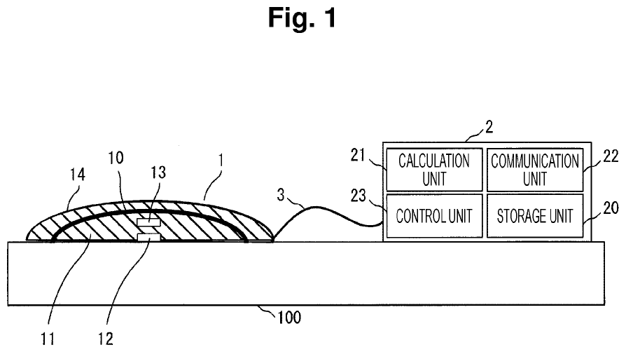

Hereinafter, embodiments of the present invention will be described with reference to the drawings. FIG. 1 is a diagram illustrating a configuration of a temperature measurement device according to a first embodiment of the present invention. The temperature measurement device includes a sensor unit 1 that measures the magnitude of a heat flow transmitted from a living body 100, and an electronic circuit 2 that calculates a core body temperature T_{cbt} of the living body 100 on the basis of the measured magnitude of the heat flow.

The sensor unit 1 includes a thermal conductor 10 having a hollow structure in which a peripheral edge portion is disposed to be in contact with the living body 100 and which has thermal conduction anisotropy and flexibility in which a thermal conductivity in an in-plane direction is higher than a thermal conductivity in a thickness direction, a heat insulating material 11 having flexibility disposed to fill a space between the living body 100 and the thermal conductor 10, a temperature sensor 12 provided on a surface of the heat insulating material 11 facing the living body 100 and configured to measure a temperature Tₛₖᵢₙ of a skin surface of the living body 100, a temperature sensor 13 configured to measure a temperature Tₜₒₚ of an inside of the heat insulating material 11 immediately above the temperature sensor 12, and a heat insulating material 14 having flexibility disposed to cover the thermal conductor 10.

The electronic circuit 2 includes a storage unit 20 that stores data, a calculation unit 21 that calculates a core body temperature T_{cbt} of the living body 100 on the basis of measurement results of the temperature sensors 12 and 13, a communication unit 22 that transmits data of the core body temperature T_{cbt} to an external terminal, and a control unit 23 that controls reading/writing of data from/in the storage unit 20 and communication.

The sensor unit 1 is mounted such that the heat insulating materials 11 and 14 and the thermal conductor 10 are in contact with the skin of the living body 100. For example, it is desirable to attach the sensor unit 1 to the living body 100 using double-sided tape or silicon rubber having excellent biocompatibility.

The thermal conductor 10 is a member having a hollow structure with an outer shape of, for example, a dome shape or a truncated cone shape. The thermal conductor 10 is disposed such that the peripheral edge portion is in contact with the living body 100. The thermal conductor 10 has thermal conduction anisotropy in which the thermal conductivity in the in-plane direction perpendicular to the thickness direction is higher than the thermal conductivity in the thickness direction, and flexibility. Such a thermal conductor 10 can be realized, for example, by orienting graphite in a plane of a polymer film to have a structure close to a single crystal. It has a thermal conductivity several times higher than that of aluminum or the like in the orientation direction.

Further, a metal thin film or metal fiber having a thickness of about several μm has flexibility. Therefore, as the thermal conductor 10, a metal thin film or a metal fiber layer and a polymer film alternately laminated may be used. The metal fiber layer is molded in a layered shape so that the metal fibers face the in-plane direction while being entangled with each other. By alternately laminating the metal thin film or the metal fiber layer and the polymer film, thermal conduction anisotropy and flexibility can be realized. In the case of using a metal thin film, patterning for removing a part may be performed to further enhance the flexibility.

The thin film thermal conductor 10 is soft. Therefore, it is difficult to maintain the entire shape of the sensor unit 1 only with the thermal conductor 10. Therefore, in a space inside the thermal conductor 10 having a hollow structure, the heat insulating material 11 is disposed to fill this space. The temperature sensor 12 is provided on the surface of the heat insulating material 11 on the living body side. The temperature sensor 13 is provided inside the heat insulating material 11 immediately above the temperature sensor 12. As the temperature sensors 12 and 13, for example, a thermistor, a thermocouple, a platinum resistor, an integrated circuit (IC) temperature sensor, or the like can be used.

The heat insulating material 11 holds the temperature sensors 12 and 13 and serves as a resistor against heat flowing into the temperature sensors 12 and 13. As a material of the heat insulating material 11, it is required to be deformed in accordance with the shape of the living body 100 while holding the temperature sensors 12 and 13, and a polymer elastic fiber, a foamed polymer, or the like can be used.

Further, the heat insulating material 14 is disposed outside the thermal conductor 10. The heat insulating material 14 is provided to maintain the shape of the sensor unit 1, block unnecessary heat flow, and protect the thermal conductor 10. Similarly to the heat insulating material 11, a polymer elastic fiber, a foamed polymer, or the like can be used as a material of the heat insulating material 14.

As described above, the sensor unit 1 has a structure in which the heat insulating material 11, the thermal conductor 10, and the heat insulating material 14 are laminated. In a case where the thermal conductor 10 is sufficiently large with respect to the temperature sensors 12 and 13, the peripheral edge portion of the thermal conductor 10 in contact with the living body 100 is disposed at a position sufficiently away from the temperature sensors 12 and 13, and thus a heat flux from the living body 100 is collected by the thermal conductor 10 outside the temperature sensors 12 and 13 and transported to the top surface of the thermal conductor 10. In this manner, the thermal conductor 10 efficiently transports the heat flux from the living body 100 upward outside the temperature sensors 12 and 13, thereby performing a function of curbing the heat flux that deviates from the temperature sensors 12 and 13 and flows out to the outside air. In addition, the thermal conductor 10 has thermal conduction anisotropy in which the thermal conductivity in the in-plane direction is higher than the thermal conductivity in the thickness direction. Therefore, it is possible to curb the transportation of heat from the thermal conductor 10 to the surroundings.

Furthermore, since the heat insulating materials 11 and 14 and the thermal conductor 10 have flexibility, they can be deformed in accordance with the shape of the living body 100. Therefore, it is easy to attach the sensor unit 1 to the living body 100. In addition, the feeling of wearing on the living body 100 can be improved, and the possibility of the living body 100 being injured by the hard device can be reduced.

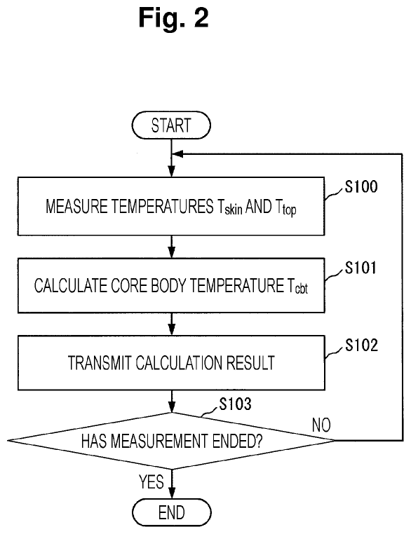

The temperature sensors 12 and 13 and the electronic circuit 2 are connected by a wire 3. FIG. 2 is a flowchart for describing the operation of the temperature measurement device of the present embodiment. The temperature sensor 12 measures the temperature Tₛₖᵢₙ of the skin surface of the living body 100. The temperature sensor 13 measures the temperature Tₜₒₚ inside the heat insulating material 11 at a position away from the living body 100 (step S100 in FIG. 2). Measurement data of the temperature sensors 12 and 13 is stored in the storage unit 20.

The calculation unit 21 calculates the core body temperature T_{cbt} (internal temperature) of the living body 100 using, for example, Expression (1) on the basis of the temperatures Tₛₖᵢₙ and Tₜₒₚ and a predetermined proportionality coefficient α (step S101 in FIG. 2).

The communication unit 22 transmits the data of the core body temperature T_{cbt} to an external terminal such as a PC or a smartphone (step S102 in FIG. 2). The external terminal displays the value of the core body temperature T_{cbt} received from the temperature measurement device.

The temperature measurement device performs the above processing of steps S100 to S102 at regular time intervals, for example, until there is an instruction to end the measurement from a user (YES in step S103 in FIG. 2).

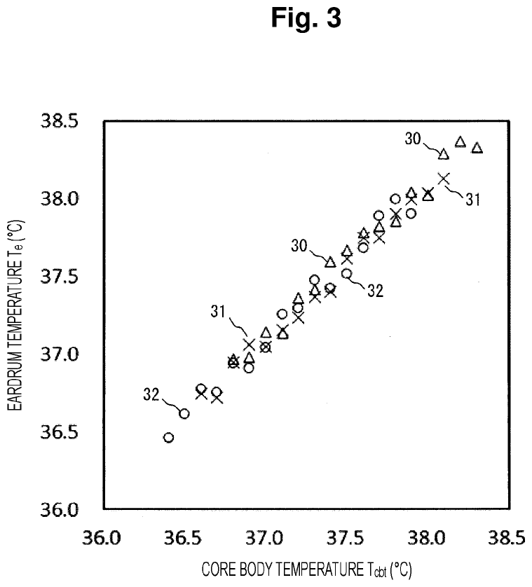

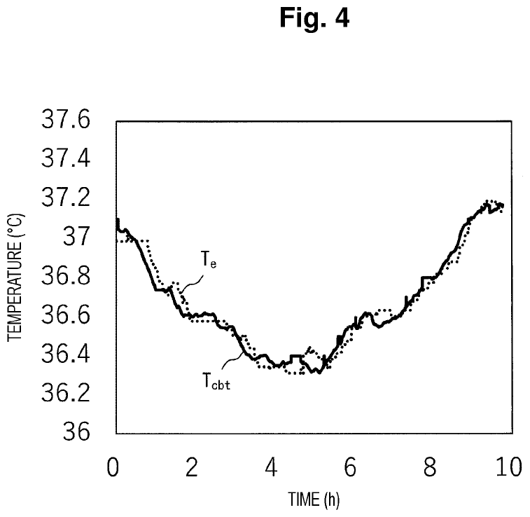

FIG. 3 illustrates a core body temperature T_{cbt} estimated in the present embodiment and a core temperature (eardrum temperature) Tₑ measured by an eardrum thermometer for comparison. Reference numerals 30, 31, and 32 in FIG. 3 represent results for different living bodies 100. In addition, FIG. 4 illustrates temporal changes in the core body temperature T_{cbt} estimated in the present embodiment and the eardrum temperature Tₑ. According to FIGS. 3 and 4, it can be ascertained that estimation results close to the eardrum temperature Tₑ are obtained according to the present embodiment.

### Second Embodiment

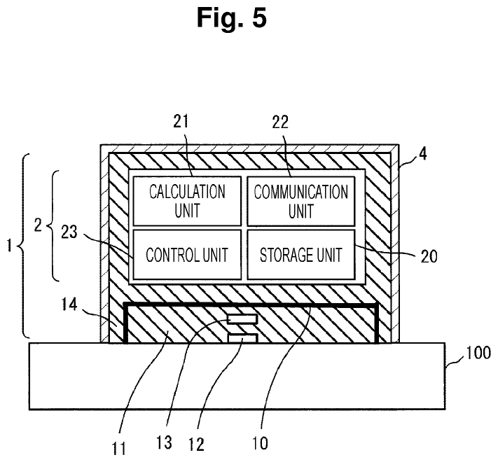

Next, a second embodiment of the present invention will be described. FIG. 5 is a diagram illustrating a configuration of a temperature measurement device according to a second embodiment of the present invention. The temperature measurement device of the present embodiment includes a sensor unit 1, an electronic circuit 2, and a radiation prevention film 4 covering the sensor unit 1 and the electronic circuit 2.

In the present embodiment, the electronic circuit 2 is provided inside a heat insulating material 14 covering a thermal conductor 10 of the sensor unit 1. In addition, in order to curb radiation of heat from the sensor unit 1 and absorption of heat from the outside, the radiation prevention film 4 is provided to cover the sensor unit 1 and the electronic circuit 2.

As the radiation prevention film 4, it is desirable to use a thin film material having a low radiation factor of heat and a high reflectance for light having wavelengths included in sunlight, and for example, an aluminum thin film can be used. In order to protect the aluminum thin film, the surface may be protected with a polymer thin film.

In the present embodiment, the mounting area of the temperature measurement device on the living body 100 can be reduced as compared with the configuration in which the sensor unit 1 and the electronic circuit 2 are separated as in the first embodiment.

### Third Embodiment

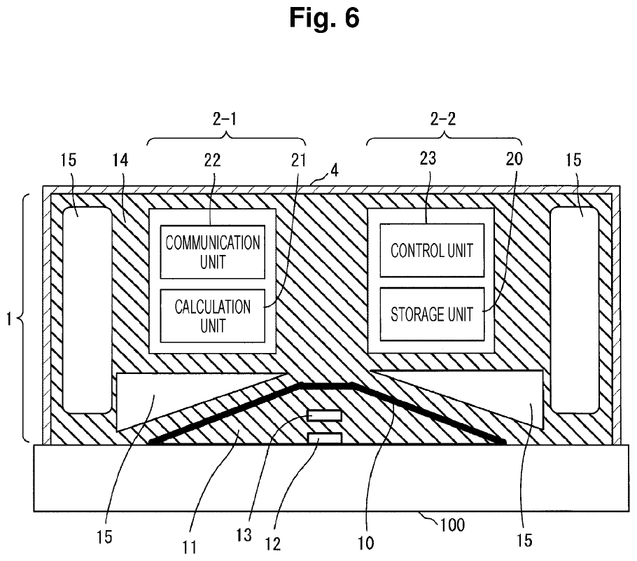

Next, a third embodiment of the present invention will be described. FIG. 6 is a diagram illustrating a configuration of a temperature measurement device according to a third embodiment of the present invention. The temperature measurement device of the present embodiment includes a sensor unit 1, electronic circuits 2-1 and 2-2, and a radiation prevention film 4 covering the sensor unit 1 and the electronic circuits 2-1 and 2-2.

In the present embodiment, similarly to the second embodiment, the electronic circuits 2-1 and 2-2 are provided inside a heat insulating material 14. Differences from the second embodiment are that the electronic circuits are provided at a plurality of locations in a distributed manner, and a space 15 is formed in the heat insulating material 14 such that the heat insulating material 14 can be more greatly deformed.

In the example of FIG. 6, the calculation unit 21 and the communication unit 22 are provided in the electronic circuit 2-1, and the storage unit 20 and the control unit 23 are provided in the electronic circuit 2-2. The manner of division in the example of FIG. 6 is an example, and another manner of division may be used. In addition, the electronic circuit may be divided into three or more.

### Fourth Embodiment

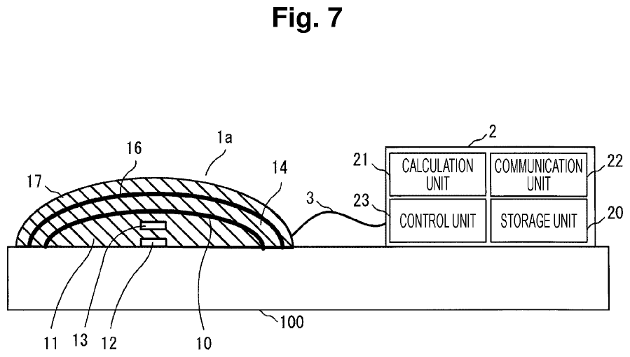

Next, a fourth embodiment of the present invention will be described. FIG. 7 is a diagram illustrating a configuration of a temperature measurement device according to a fourth embodiment of the present invention. The temperature measurement device of the present embodiment includes a sensor unit 1a and an electronic circuit 2.

The sensor unit 1a of the present embodiment includes a thermal conductor 10, a heat insulating material 11, temperature sensors 12 and 13, a heat insulating material 14, a thermal conductor 16, and a heat insulating material 17 having flexibility disposed to cover the thermal conductor 16.

The thermal conductor 16 is made of the same material as the thermal conductor 10. The thermal conductor 16 is disposed such that the peripheral edge portion thereof is in contact with the living body 100 and covers the heat insulating material 14.

As described above, the sensor unit 1a of the present embodiment has a structure in which the heat insulating material 11, the thermal conductor 10, the heat insulating material 14, the thermal conductor 16, and the heat insulating material 17 are laminated. By alternately laminating the heat insulating materials 11, 14, and 17 and the thermal conductors 10 and 16, heat transport from the thermal conductors 10 and 16 to the surroundings can be further curbed. Although two layers of the thermal conductors 10 and 16 and two layers of the heat insulating materials 14 and 17 are provided in the example of FIG. 7, three or more layers of thermal conductors and heat insulating materials may be provided.

The configuration of the electronic circuit 2 is the same as that of the first embodiment. The configurations of the second and third embodiments may be applied to the present embodiment. In this case, the electronic circuits 2, 2-1, and 2-2 and the space 15 are provided inside the heat insulating material 17.

In addition, in the first to fourth embodiments, the electronic circuits 2, 2-1, and 2-2 can be deformed by using a flexible material such as polyimide for substrates of the electronic circuits 2, 2-1, and 2-2.

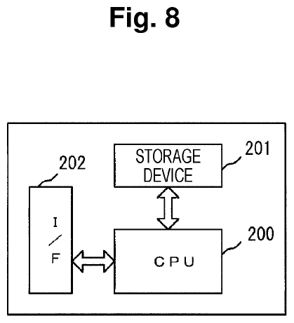

The storage unit 20, the calculation unit 21, the communication unit 22, and the control unit 23 described in the first to fourth embodiments can be realized by a computer including a central processing unit (CPU), a storage device, and an interface, and a program for controlling these hardware resources. A configuration example of this computer is illustrated in FIG. 8.

The computer includes a CPU 200, a storage device 201, and an interface device (I/F) 202. Hardware or the like of the temperature sensors 12 and 13 and the communication unit 22 is connected to the I/F 202. In such a computer, a program for realizing a temperature measurement method of embodiments of the present invention is stored in the storage device 201. The CPU 200 executes processing described in the first to fourth embodiments according to a program stored in the storage device 201.

### INDUSTRIAL APPLICABILITY

Embodiments of the present invention can be applied to a technique for noninvasively measuring the internal temperature of a living body.

### REFERENCE SIGNS LIST

1, la Sensor unit
2, 2-1, 2-2 Electronic circuit
3 wire
4 Radiation prevention film
10, 16 Thermal conductor
11, 14, 17 Heat insulating material
12, 13 Temperature sensor
15 Space
20 Storage unit
21 Calculation unit
22 Communication unit
23 Control unit

1-7. (canceled)

8. A temperature measurement device comprising:
a thermal conductor having a hollow structure, a peripheral edge portion of the thermal conductor being configured to be disposed in contact with a living body, the thermal conductor having thermal conduction anisotropy in which a thermal conductivity in an in-plane direction is higher than a thermal conductivity in a thickness direction, the thermal conductor further having flexibility;
a first heat insulating material having flexibility, the first heat insulating material being configured to fill a space between the living body and the thermal conductor;
a sensor on the first heat insulating material and configured to measure a magnitude of a heat flow transmitted from the living body;
a second heat insulating material having flexibility, the second heat insulating material covering the thermal conductor; and
an electronic circuit configured to calculate an internal temperature of the living body based on the magnitude of the heat flow measured by the sensor.

9. The temperature measurement device according to claim 8, wherein the electronic circuit is provided inside the second heat insulating material.

10. The temperature measurement device according to claim 9, wherein
the electronic circuit is provided at a plurality of locations in a distributed manner inside the second heat insulating material.

11. The temperature measurement device according to claim 8, wherein
a space is disposed inside the second heat insulating material.

12. The temperature measurement device according to claim 8, wherein
a plurality of the thermal conductors and a plurality of the second heat insulating materials are alternatingly laminated.

13. The temperature measurement device according to claim 8, further comprising a film configured to reduce radiation of heat, the film being provided to cover an outer side of the second heat insulating material.

14. The temperature measurement device according to claim 8, wherein the sensor includes:
a first temperature sensor on a surface of the first heat insulating material facing the living body and configured to measure a temperature of a surface of the living body; and
a second temperature sensor configured to measure a temperature inside the first heat insulating material immediately above the first temperature sensor, wherein the electronic circuit is configured to calculate the internal temperature of the living body based on measurement results of the first and second temperature sensors.

15. A temperature measurement method comprising:
providing temperature measurement device comprising:
a thermal conductor having a hollow structure, a peripheral edge portion of the thermal conductor being disposed in contact with a living body, the thermal conductor having thermal conduction anisotropy in which a thermal conductivity in an in-plane direction is higher than a thermal conductivity in a thickness direction, the thermal conductor further having flexibility;
a first heat insulating material having flexibility, the first heat insulating material filling a space between the living body and the thermal conductor;
a sensor on the first heat insulating material;
a second heat insulating material having flexibility, the second heat insulating material covering the thermal conductor; and
an electronic circuit;
measuring, by the sensor, a magnitude of a heat flow transmitted from the living body; and
calculating an internal temperature of the living body based on the magnitude of the heat flow measured by the sensor.

16. The temperature measurement method according to claim 15, wherein the electronic circuit is provided inside the second heat insulating material.

17. The temperature measurement method according to claim 16, wherein
the electronic circuit is provided at a plurality of locations in a distributed manner inside the second heat insulating material.

18. The temperature measurement method according to claim 15, wherein
a space is disposed inside the second heat insulating material.

19. The temperature measurement method according to claim 15, wherein
a plurality of the thermal conductors and a plurality of the second heat insulating materials are alternatingly laminated.

20. The temperature measurement method according to claim 15, wherein the temperature measurement device further comprises a film configured to reduce radiation of heat, the film being provided to cover an outer side of the second heat insulating material.
